(19) United States
(12) Patent Application Publication
(10) Pub. No.: US 2026/0174348 A1
(43) Pub. Date: Jun. 25, 2026

(54) SYSTEMS AND METHODS FOR AN IMPROVED PRESSURE SENSING CANNULA

(71) Applicant: Regional Perfusion Services LLC, Richardson, TX (US)

(72) Inventors: Murphy Rayle, Richardson, TX (US); Scott Noesges, Fairview, TX (US)

(21) Appl. No.: 18/987,566

(22) Filed: Dec. 19, 2024

(51) Int. Cl. A61B 5/03; A61M 1/16; A61M 1/36

(52) U.S. Cl. CPC A61B 5/03; A61M 1/1698; A61M 1/3641; A61M 1/3659

(57) ABSTRACT

A cannula system includes a main cannula, the main cannula providing blood exchange and a lumen isolated from the main cannula, the lumen providing a means for measuring pressure in an area of a human body.

### BACKGROUND

In many scenarios, the organs of patients may not receive sufficient oxygen, pressure, and warmth. This may occur when a patient is injured, undergoing surgery, in a coma, or brain dead. The health and viability of the organs may suffer in such a scenario and either may case issues for the patient or prevent transplant of organs. A better technique to maintain organs is desirable.

### BRIEF SUMMARY

In one embodiment, a cannula system includes a main cannula, the main cannula providing blood exchange and a lumen isolated from the main cannula, the lumen providing a means for measuring pressure in an area of a human body. Alternatively, the main cannula includes a distal end, the distal end allowing for fluid to flow from the main cannula. In one alternative, the lumen includes an open end, the open end of the lumen set back from the distal end of the main cannula a distance effective for limiting an effect of fluid flow from the distal end of the main cannula on pressure sensed via the lumen. In another alternative, the cannula system includes a tube, the tube molded in the main cannula. In another alternative, the cannula system includes a stylet, the stylet located at the distal end of the main cannula. Alternatively, the stylet has a tapered exit. In one alternative, the cannula has a lure lock connector on a proximate end of the cannula. Alternatively, the distance effective for limiting the effect of fluid flow is proportional to the size of the tapered exit. In another alternative, the distance is 1.5 cm. Alternatively, the lumen is fused in fused in polypropylene to the main cannula. In another alternative, the main cannula is interconnected with a heater/oxygenator. Alternatively, the main cannula is interconnected to a pumping system. In another alternative, the pumping system is a centrifugal pump.

In one embodiment, a method of increasing life of organs of a patient includes providing an improved cannula, the improved cannula including: a main cannula, the main cannula providing blood exchange and a lumen isolated from the main cannula, the lumen providing a means for measuring pressure in an area of a human body. The method further includes using the main cannula to exchange blood near organs of the patient and monitoring the pressure in the area of the human body using the lumen, the human body belonging to the patient. In one alternative, the method includes adjusting the pressure in the area of the human body using a transducer, connected to the improved cannula. In one alternative, the method includes oxygenating and heating the blood provided to the main cannula, wherein the blood is maintained at an oxygen and a temperature to maintain the organs of the patient. In another alternative, the method includes isolating the organs of the patient using a clamp. Alternatively, the isolating using the clamp incudes a technique selected from the list consisting of clamping thoracic artery and clamping NRP.

In one embodiment an improved cannula includes a main cannula, the main cannula providing blood exchange and a lumen isolated from the main cannula, the lumen providing a means for measuring pressure in an area of a human body. The main cannula includes a distal end, the distal end allowing for fluid to flow from the main cannula and the lumen includes an open end, the open end of the lumen set back from the distal end of the main cannula a distance effective for limiting an effect of fluid flow from the distal end of the main cannula on pressure sensed via the lumen. In one alternative, the improved cannula includes a tube, the tube molded in the main cannula and a stylet, the stylet located at the distal end of the main cannula, wherein the stylet has a tapered exit.

### BRIEF DESCRIPTION OF THE DRAWINGS

FIG. 1 shows one embodiment of a system for Normothermic Regional Perfusion (NRP) including an improved cannula;

FIG. 2 shows one embodiment of an improved pressure sensing cannula;

FIG. 3 shows another view of the improved pressure sensing cannula of FIG. 2;

FIG. 3 shows another view of the improved pressure sensing cannula of FIG. 2;

FIG. 4 shows another view of the improved cannula of FIG. 2;

FIG. 5 shows an embodiment of an improved cannula;

FIG. 6 shows an embodiment of an improved cannula;

FIG. 7 shows a detail view of the tip of cannula of FIG. 6;

FIG. 8 shows another view of a cannula;

FIG. 9 shows improved cannula; and

FIGS. 10-12 show examples of the improved cannula deployed in patients.

### DETAILED DESCRIPTION

Certain terminology is used herein for convenience only and is not to be taken as a limitation on the embodiments of the systems and methods for an improved pressure sensing cannula.

In some embodiments, an improved pressure sensing cannula is a wire-reinforced arterial perfusion cannula used to supply 1-6 LPM of blood flow to an isolated region of the body. In many embodiments, the improved pressure sensing cannula is used as part of a Normothermic Regional Perfusion (NRP) cases; the cannula is a conduit for the arterial vessel so oxygenated blood can be reinfused to targeted donor organs for transplant. Other applications for this cannula include, cardiac bypass surgery, and extra-corporeal membrane oxygenation (ECMO) and much more.

Numerous configurations of embodiments of an improved pressure sensing cannula are possible, including various sizes, materials and shapes. In some embodiments, the cannula will range in size from 4.5 mm (13 fr) 5 mm (15 fr), 6mm(18fr), 7 mm (21 fr), and 8 mm (24 fr) in cannula diameter. It will have a ¼th—inch plastic connector for the 4.5 mm (13 fr) cannula and a ⅜th-inch plastic connector for the remaining cannulas, to be bonded at the back of the cannula. In some configurations, off the side of the cannula, a secondary tube that runs in the internal lumen of the cannula and exits out of the tip 1 cm back from the distal opening of the cannula. In some designs, this tube is molded in the internal or external wall of the cannula, approximately 0.25 mm in diameter into the center of the cannula and adhered to the internal or external wall of the cannula. In some scenarios, the tube is 16 cm in length and an 18 gauge stylet integrated into the cannula. Optionally, the stylet has a simple tapered exit from the distal end of the cannula and has a lure lock connector on the proximal end of the cannula on the ⅜th connector. In some alternatives, a lure lock connects to a long-primed pressure transducer line, for the purpose of measuring the intra-vascular arterial pressure. Through this novel application, mean arterial pressure could be monitored without the necessity of additional interventions to the patient/donor.

In many embodiments, a cannula is to be inserted after the Declaration of Circulatory Death (DCD) and is utilized to capture the systemic regional arterial pressure of targeted organs for transplant. This allows Normothermic Regional Perfusion (NRP) providers to accurately access the pressure of the isolated region, to determine appropriate mechanical or pharmacological support to optimize the reperfusion of organs for transplant. Specifically, utilized to recognize optimal mean arterial pressure (MAP) to ensure organ perfusion is appropriate and in optimal physiologic range for organ function. Various metrics are monitored and controlled, including, but not limited to Kidney perfusion to optimize for urine output, GFR optimization, reduction of hypotension/hypertensive effects on organ (heart, lungs, liver, kidney) function, and appropriate interpretation of systemic vascular resistance.

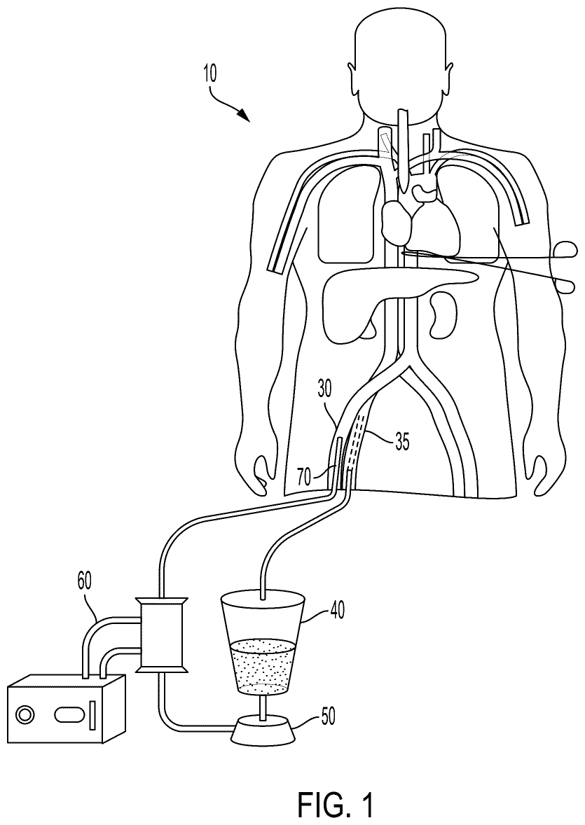

FIG. 1 shows one embodiment of a system for Normothermic Regional Perfusion (NRP) including an improved cannula. Patient 10 in many scenarios, has been incapacitated, dying, or is clinically dead. It is desirable to maintain and preserve the organs of patient 10. In order to accomplish this, it is necessary to provide continuous heated and oxygenated blood. This is performed via NRP. First, in many scenarios, an aortic clamp 20 is utilized to isolate the heart of patient 10. Once aortic clamp 20 is applied, the NRP system is attached to the user via arteries 30, 35. Blood is removed from patient 10 (via a tube or other mechanism) and transported to reservoir 40. Centrifugal pump 50 provides pressure to pump the blood to heater/oxygenator 60. At heater/oxygenator 60 the blood is heated and oxygenated in order to maintain organ function, thus extending the possibility of transplant for a period of time. The blood returns to the patient 10 via cannula 70. Cannula 70 is a specialized cannula, also subject of the current disclosure. Cannula 70 includes a pressure sensing mechanism to provides feedback to centrifugal pump 50 in order to maintain sufficient pressure in the patient 10 to maintain organ function and life.

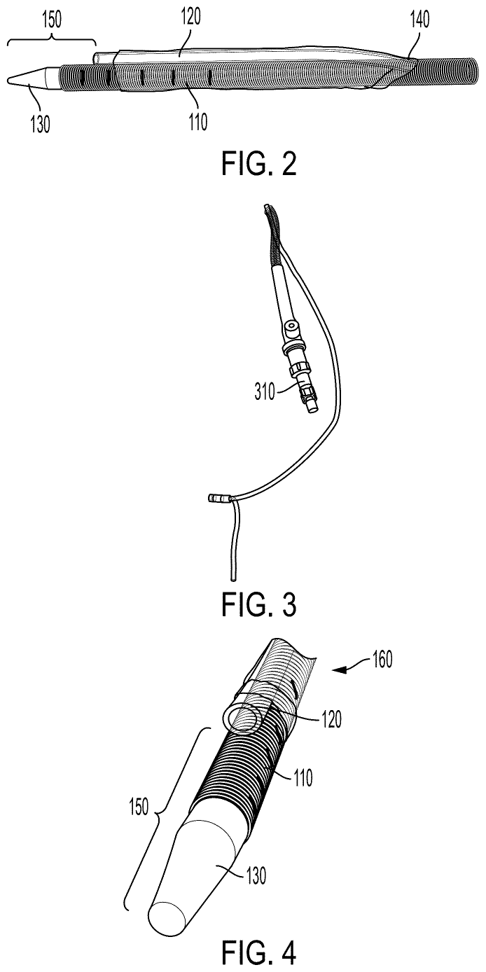

FIG. 2 shows one embodiment of an improved pressure sensing cannula 100. Improved pressure sensing cannula 100 includes main cannula 110. Main cannula 110 provides for the flow of replenished blood to the patient. Lumen 120 is attached or formed in conjunction with main cannula 110. There are many possibilities for the attachment and formation of lumen 120. Lumen 120 may be part of a single piece of material, such as plastic, formed in conjunction with the main cannula 110. Alternatively, lumen 120 may be later attached or bonded to main cannula 110 via a variety of method. [include examples of both of these.] Lumen 120 includes a secondary tube 140 that runs in the internal lumen 120 of the cannula 100 and exits out of the tip a distance 150 of 1 cm back from the distal opening. This tube is molded in the internal or external wall of the cannula, approximately 0.25 mm in diameter into the center of the cannula and adhered to the internal or external wall of the cannula. Distance 150 may differ according to many factors but is generally designed to prevent the effects of turbulence that exits from the main cannula 110 that would affect pressure readings.

Main cannula 110 include distal end where a stylus 130 is located. Stylus 130 may take a variety of forms and sizes but is typically conical in nature. Main cannula 110 typically ranges in size from 4.5 mm (13 fr) 5 mm (15 fr), 6 mm (18 fr), 7 mm (21 fr), and 8 mm (24 fr) in cannula diameter. Cannula 110 includes a ¼th—inch plastic connector 310 for the 4.5 mm (13 fr) cannula and a ⅜th-inch plastic connector for the remaining cannulas, to be bonded at the back of the cannula. The plastic connector 310 is shown in FIG. 3 and may vary in size and design according to various use specifications. The secondary pressure lumen 18ga or 20ga & 1.5-2 cm shorter than cannula length.

FIG. 4 shows another view of cannula 110. Main cannula 110, lumen 120, stylus 130 are shown arranged with lumen 120 offset distance 150 from the distal end of main cannula 110.

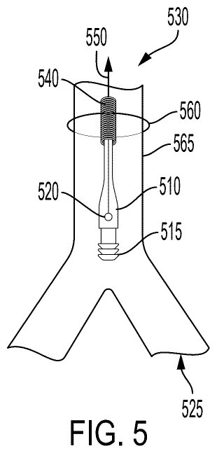

FIG. 5 shows an embodiment of an improved cannula 510. In this view, cannula 510 is deployed int the arteries of a patient. As shown, end 515 is interconnected to a NRP circuit. Additionally, connector 520 is connected to a pressure line attached to a pressure transducer. The iliac artery 525 may feed into The cannula 510 may be deployed in region of blood flow 530. Via cannula 510, the pressure of aorta 565 is transduced 540. Umbilical tape tie 560 may be deployed in many setups in order to occlude portions of the patient's system.

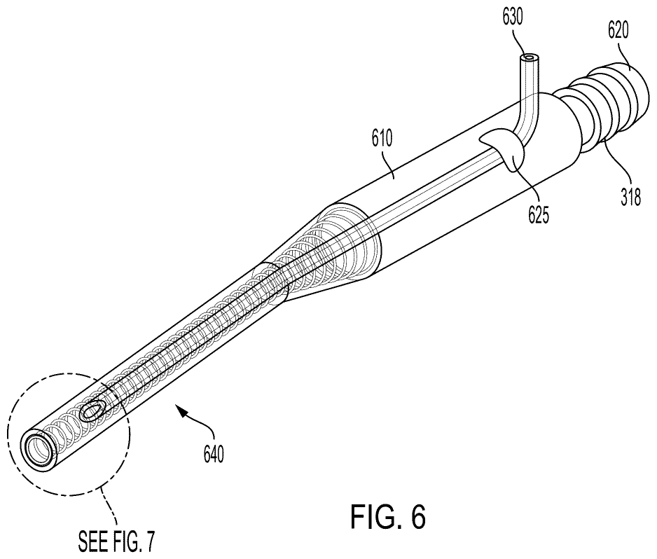

FIG. 6 shows an embodiment of an improved cannula 610. Here the general tubular structure of the cannula 610 can be seen. In this configuration, cannula 610 includes a connector 620. In many embodiments, this is a ⅜ inch connector. Numerous other size connectors are possible. The cannula 610 also includes a lure connector 630. In many configurations, the lure connector 630 is fused in polypropylene to the cannula 610. In many embodiments, cannula 610 is reinforced with a wire frame 640. This helps maintain the structure of the cannula 610 and prevents collapse.

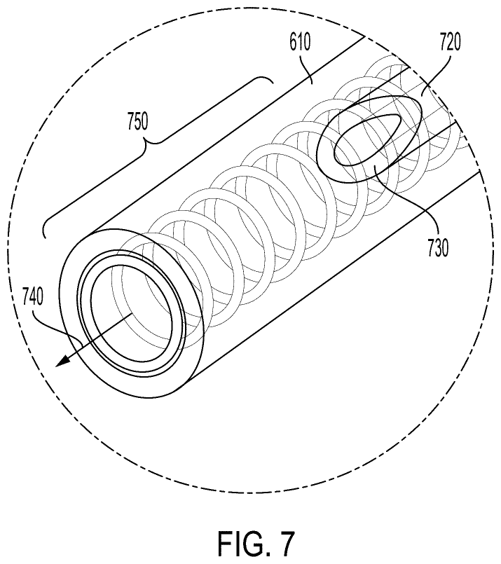

FIG. 7 shows a detail view of the tip of cannula 610. Shown here is the secondary tube 720 that is interconnected to a pressure measuring transducer. Tip 730 is molded on the exterior of cannula 610. The tip 730 is located a distance 750 from the end of the cannula 740, through which blood flows out from. Distance 750 in some embodiments is 1.5 cm, however this distance may vary depending on the configuration.

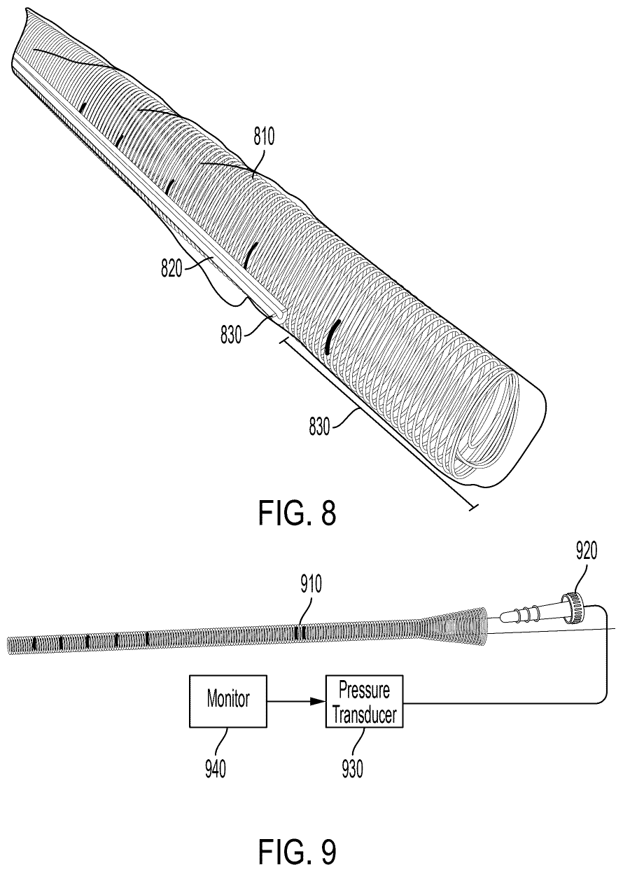

FIG. 8 shows another view of a cannula 810. In this view the secondary tube 820 is show with beveled end 830. In many embodiments, the secondary tube includes a 18 g pressure line. As shown, beveled end 830 is a distance 840 from the end of the cannula 810. In many embodiments, distance 840 is 1.5 cm.

FIG. 9 shows improved cannula 910 connected for use. Lure connection 920 is connected to pressure transducer 930, which enables the regulation of pressure in the patient body. Monitor 940 provide pressure readings and may include a modulation algorithm for control of pressure transducer 930 as well as provisions for manual control.

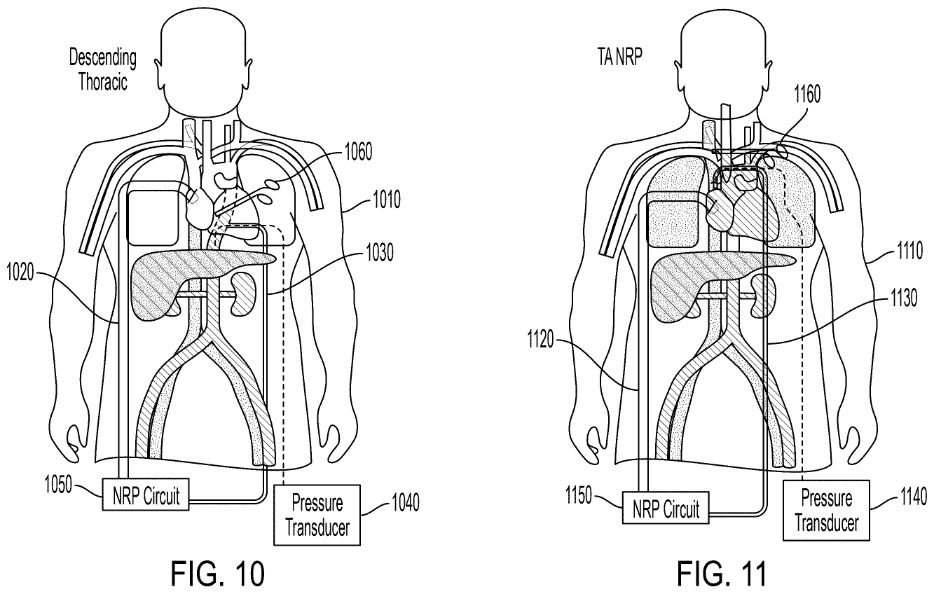

FIGS. 10-12 show examples of the improved cannula deployed in patients. In each scenario, the condition of the patient varies, and the intent is to maintain different organs of the patient. In FIG. 10, a descending thoracic artery has been clamped 1060 in patient 1010. In such a scenario, the liver and kidneys are primarily maintained in the patient. In such a scenario, NRP circuit (Normothermic Regional Perfusion) 1050 is functionally connected via tubes 1020. Blood circulates out of the system via tube 1030 and improved cannula. Pressure transducer 1040, monitors and maintains the pressure of the system.

Similarly, in FIG. 11, a Thoracoabdominal NRP has been clamped 1160 in patient 1110. In such a scenario, the liver and kidneys and lungs and heart are primarily maintained in the patient. In such a scenario, NRP circuit (Normothermic Regional Perfusion) 1150 is functionally connected via tubes 1120. Blood circulates out of the system via tube 1130 and improved cannula. Pressure transducer 1140, monitors and maintains the pressure of the system.

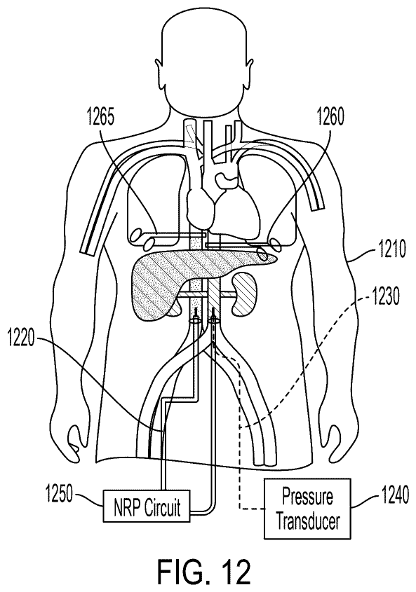

Similarly, in FIG. 12, a lower area has been clamped 1260 in patient 1210. In such a scenario, the liver and kidneys are primarily maintained in the patient. In such a scenario, NRP circuit (Normothermic Regional Perfusion) 1250 is functionally connected via tubes 1220. Blood circulates out of the system via tube 1230 and improved cannula. Pressure transducer 1240, monitors and maintains the pressure of the system.

By maintaining the flow, oxygenation, temperature, a pressure of the blood the various organs as discussed in FIGS. 10-12 may be maintained for a longer period of time for transplant.

In many configurations, centrifugal flow in the cannula is a direct measurement from an integrated or stand-alone probe that specifically measures the flow. Cannulas based on design from tip design to taper can change; this is not a direct measurement but can be utilized via an indirect measurement. The cannula based on an open-ended tip design will have less pressure drop across the tip (p1v1=p2v2), leading to a relative flow and pressure rating. With this information, an individual can deduct relative pressure without a direct measurement.

In many embodiments, an enhanced cannula system is provided offering a blood exchange system and a pressure sensing system in a cannula. In normal circumstances, when blood exchange is occurring, the patient is sufficiently alive, so the pressure is not an issue. With a deceased or otherwise incapacitated individual, pressure control may be more of an issue. Under normal circumstances Perfusionists work with living patients. Therefore, the pressure is not as much of an issue. But when reframed in the context of the maintenance of organs in a dead patient, then the invention is needed.

Application of the improved lumen and techniques herein may be applied in a variety of scenarios. These scenarios include: 1) lack of neural input, affects of the brain on vasoconstriction/vasodilation; 2) anatomical anomalies, ability to access; 3) aortic dissection and interpretation; 4) Improper cannula location-help detect if cannula is in a false lumen; and 5) Lack of vasculature in targeted regions (patients that have expired)—living patients have access in multiple locations for pressure monitoring, after a patient expires and NRP is conducted, those points of pressure access are now isolated out.

While specific embodiments have been described in detail in the foregoing detailed description, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof.

What is claimed as new and desired to be protected by Letters Patent of the united States is:

1. A cannula system, comprising:
a main cannula, the main cannula providing blood exchange;
a lumen isolated from the main cannula, the lumen providing a means for measuring pressure in an area of a human body.

2. The cannula system of claim 1, wherein the main cannula includes a distal end, the distal end allowing for fluid to flow from the main cannula.

3. The cannula system of claim 2, wherein the lumen includes an open end, the open end of the lumen set back from the distal end of the main cannula a distance effective for limiting an effect of fluid flow from the distal end of the main cannula on pressure sensed via the lumen.

4. The cannula system of claim 3, further comprising: a tube, the tube molded in the main cannula.

5. The cannula system of claim 4, further comprising: a stylet, the stylet located at the distal end of the main cannula.

6. The cannula system of claim 5, wherein the stylet has a tapered exit.

7. The cannula system of claim 6, wherein the cannula has a lure lock connector on a proximate end of the cannula.

8. The cannula system of claim 7, wherein the distance effective for limiting the effect of fluid flow is proportional to the size of the tapered exit.

9. The cannula system of claim 8, wherein the distance is 1.5 cm.

10. The cannula system of claim 9, wherein the lumen is fused in fused in polypropylene to the main cannula.

11. The cannula system of claim 10, wherein the main cannula is interconnected with a heater/oxygenator.

12. The cannula system of claim 11, wherein the main cannula is interconnected to a pumping system.

13. The cannula system of claim 12, wherein the pumping system is a centrifugal pump.

14. A method of increasing life of organs of a patient, the method comprising:
providing an improved cannula, the improved cannula including:
a main cannula, the main cannula providing blood exchange;
a lumen isolated from the main cannula, the lumen providing a means for measuring pressure in an area of a human body;
using the main cannula to exchange blood near organs of the patient;
monitoring the pressure in the area of the human body using the lumen, the human body belonging to the patient.

15. The method of claim 14, further comprising:
adjusting the pressure in the area of the human body using a transducer, connected to the improved cannula.

16. The method of claim 15, further comprising:
oxygenating and heating the blood provided to the main cannula, wherein the blood is maintained at an oxygen and a temperature to maintain the organs of the patient.

17. The method of claim 16, further comprising:
isolating the organs of the patient using a clamp.

18. The method of claim 13, wherein the isolating using the clamp incudes a technique selected from the list consisting of clamping thoracic artery and clamping thoracoabdominal NRP.

19. An improved cannula, the improved cannula comprising:
a main cannula, the main cannula providing blood exchange;
a lumen isolated from the main cannula, the lumen providing a means for measuring pressure in an area of a human body; wherein the main cannula includes a distal end, the distal end allowing for fluid to flow from the main cannula and the lumen includes an open end, the open end of the lumen set back from the distal end of the main cannula a distance effective for limiting an effect of fluid flow from the distal end of the main cannula on pressure sensed via the lumen.

20. The improved cannula of claim 19, further comprising:
a tube, the tube molded in the main cannula;
a stylet, the stylet located at the distal end of the main cannula, wherein the stylet has a tapered exit.
